(19) United States
(12) Patent Application Publication
(10) Pub. No.: US 2003/0186435 A1
(43) Pub. Date: Oct. 2, 2003

(54) Methods of modulating FabH activity

(75) Inventors: Alexandros K. Konstantinidis, King of Prussia, PA (US); John Timothy Lonsdale, Exton, PA (US); Glenn Scott Van Aller, Reading, PA (US)

Correspondence Address: GLAXOSMITHKLINE, Corporate Intellectual Property - UW2220, P.O. Box 1539, King of Prussia, PA, 19406-0939, US

(73) Assignees: SmithKline Beecham Corporation; SmithKline Beecham p.l.c.

(21) Appl. No.: 10/443,432

(22) Filed: May 22, 2003

Related U.S. Application Data
(63) Continuation of application No. 09/980,875, filed on Oct. 29, 2001.
Application No. 09/980,875 is a 371 of international application No. PCT/US00/12250, filed on May 4, 2000.
(60) Provisional application No. 60/132,714, filed on May 6, 1999.

(51) Int. Cl. C12Q 1/68; A61K 39/12; C12N 5/06; C12N 5/16

(52) U.S. Cl. 435/345; 435/6; 424/186.1

(57) ABSTRACT

The invention provides FabH polypeptides and polynucleotides encoding FabH polypeptides and methods for producing such polypeptides by recombinant techniques. Also provided are methods for utilizing FabH polypeptides to screen for antibacterial compounds.

### FIELD OF THE INVENTION

This invention relates to newly identified mechanisms of action of FabH polypeptides. In particular, the invention relates to exploiting such mechanisms to treat disease.

### BACKGROUND OF THE INVENTION

It is particularly preferred to employ staphylococcal genes and gene products as targets for the development of antibiotics. The Staphylococci make up a medically important genera of microbes. They are known to produce two types of disease, invasive and toxigenic. Invasive infections are characterized generally by abscess formation effecting both skin surfaces and deep tissues. S. aureus is the second leading cause of bacteremia in cancer patients. Osteomyelitis, septic arthritis, septic thrombophlebitis and acute bacterial endocarditis are also relatively common. There are at least three clinical conditions resulting from the toxigenic properties of Staphylococci. The manifestation of these diseases result from the actions of exotoxins as opposed to tissue invasion and bacteremia. These conditions include: staphylococcal food poisoning, scalded skin syndrome and toxic shock syndrome.

The frequency of Staphylococcus aureus infections has risen dramatically in the past few decades. This has been attributed to the emergence of multiply antibiotic resistant strains and an increasing population of people with weakened immune systems. It is no longer uncommon to isolate Staphylococcus aureus strains which are resistant to some or all of the standard antibiotics. This phenomenon has created a demand for both new anti-microbial agents, vaccines, and diagnostic tests for this organism.

The streptococci make up a medically important genera of microbes known to cause several types of disease in humans, including, for example, otitis media, conjunctivitis, pneumonia, bacteremia, meningitis, sinusitis, pleural empyema and endocarditis, and most particularly meningitis, such as for example infection of cerebrospinal fluid. Since its isolation more than 100 years ago, Streptococcus pneumoniae has been one of the more intensively studied microbes. For example, much of our early understanding that DNA is, in fact, the genetic material was predicated on the work of Griffith and of Avery, Macleod and McCarty using this microbe. Despite the vast amount of research with S. pneumoniae, many questions concerning the virulence of this microbe remain. There is an umet medical need to employ streptococcal genes and gene products as targets for the development of antibiotics.

The pathway for the biosynthesis of saturated fatty acids is very similar in prokaryotes and eukaryotes. However, whilst the chemical reactions may not vary, the organization of the biosynthetic apparatus is very different. Vertebrates and yeasts possess type I fatty acid synthases (FASs) in which all of the enzymatic activities are encoded on one or two polypeptide chains, respectively. The acyl carrier protein (ACP) is an integral part of the complex. In contrast, in most bacterial and plant FASs (type II) each of the reactions are catalyzed by distinct monofunctional enzymes and the ACP is a discrete protein. Mycobacteria are unique in that they possess both type I and II FASs; the former is involved in basic fatty acid biosynthesis whereas the latter is involved in synthesis of complex cell envelope lipids such as mycolic acids. There therefore appears to be considerable potential for selective inhibition of the bacterial systems by broad spectrum antibacterial agents (Rock, C. & Cronan, J. 1996. Biochimica et Biophysica Acta 1302, 1-16; Jackowski, S. 1992. In Emerging Targets in Antibacterial and Antifungal Chemotherapy. Ed. J. Sutcliffe & N. Georgopapadakou. Chapman & Hall, New York; Jackowski, S. et al. (1989). J. Biol. Chem. 264, 76247629.)

The first step in the biosynthetic cycle is the condensation of malonyl-ACP with acetyl-CoA by FabH. Prior to this, malonyl-ACP is synthesized from ACP and malonyl-CoA by FabD, malonyl CoA:ACP transacylase. In subsequent rounds malonyl-ACP is condensed with the growing-chain acyl-ACP (FabB and FabF, synthases I and II respectively). The second step in the elongation cycle is ketoester reduction by NADPH-dependent β-ketoacyl-ACP reductase (FabG). Subsequent dehydration by β-hydroxyacyl-ACP dehydrase (either FabA or FabZ) leads to trans-2-enoyl-ACP which is in turn converted to acyl-ACP by NADH-dependent enoyl-ACP reductase (FabI). Further rounds of this cycle, adding two carbon atoms per cycle, eventually lead to palmitoyl-ACP whereupon the cycle is stopped largely due to feedback inhibition of FabH and 1 by palmitoyl-ACP (Heath, et al, (1996), J. Biol. Chem. 271, 1833-1836).

Cerulenin and thiolactomycin are potent and selective inhibitors of bacterial fatty acid biosynthesis. Extensive work with these inhibitors in Gram-negative bacteria has proved that this biosynthetic pathway is essential for viability. Little work has been carried out in Gram-positive bacteria.

Clearly, there exists a need for modulators of FabH polypeptide activity, particularly those useful to treat infection, dysfunction and disease.

### SUMMARY OF THE INVENTION

The invention provides compounds that modulate an activity or expression of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4.

The invention further provides a method for the treatment of an individual having need to inhibit FabH polypeptide comprising the steps of: administering to the individual a antibacterially effective amount of an antagonist that inhibits an activity or expression of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4.

Also provided is a method for the treatment of an individual infected with a bacteria comprising the steps of: administering to the individual a antibacterially effective amount of an antagonist that inhibits an activity or expression of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4.

Further provided is a method for the treatment of an individual having need to inhibit FabH polypeptide comprising the steps of: administering to the individual a antibacterially effective amount of an antagonist that inhibits a conversion of acetylCoA to product or a conversion of malonyl-ACP to product.

The invention also provides a method for the treatment of an individual infected with a bacterium having comprising the steps of: administering to the individual a antibacterially effective amount of an antagonist that inhibits a conversion of acetyl-CoA by FabH to product or a conversion of malonyl-ACP by FabH to product.

Also provided is a method for the treatment of an individual infected by a bacteria comprising the steps of: administering to the individual a antibacterially effective amount of an antagonist that inhibits a conversion of acetyl-CoA to product by Staphylococcus aureus FabH or a conversion of malonyl-ACP to product by Staphylococcus aureus FabH. Still further provided is a method for the treatment of an individual infected by Streptococcus pneumoniae comprising the steps of: administering to the individual a antibacterially effective amount of an antagonist that inhibits a conversion of acetyl-CoA to product by Streptococcus pneumoniae FabH or a conversion of malonyl-ACP to product by Streptococcus pneumoniae FabH.

Yet another method provides an antagonist that inhibits an activity of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4, wherein said activity is a conversion of acetyl-CoA to product or a conversion of malonyl-ACP to product.

This invention provides another method for the treatment of an individual having need to inhibit FabH polypeptide comprising the steps of: administering to the individual a antibacterially effective amount of an antagonist that inhibits an activity of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4, wherein said activity is a conversion of acetylCoA to product or a conversion of malonyl-ACP to product.

Also provided by the invention is a method for the treatment of an individual infected with a bacteria comprising the steps of: administering to the individual a antibacterially effective amount of an antagonist that inhibits an activity of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4 wherein said activity is a conversion of acetyl-CoA to product or a conversion of malonyl-ACP to product.

A method for inhibiting a FabH polypeptide comprising the steps of: contacting a composition comprising said polypeptide with an amount effective amount of an antagonist that inhibits a conversion of acetyl-CoA to product or a conversion of malonyl-ACP to product is also provided by the invention.

A method for inhibiting a conversion of acetyl-CoA to product or a conversion of malonyl-ACP to product comprising the steps of: contacting a composition comprising bacteria with a compound that inhibits a conversion of acetyl-oA to product or a conversion of malonyl-ACP to product for an effective time to cause killing or slowing or of growth of said bacteria is also provided herein.

The invention also provides a method for inhibiting a growth of bacteria comprising the steps of: contacting a composition comprising bacteria with an antibacterially effective amount of an antagonist that inhibits a conversion of acetyl-CoA to product by Streptococcus pneumoniae or Staphylococcus aureus FabH or a conversion of malonyl-ACP to product by Streptococcus pneumoniae or Staphylococcus aureus FabH.

A method is also provided by the invention for inhibiting a FabH polypeptide comprising the steps of: contacting a composition comprising bacteria with an antibacterially effective amount of an antagonist that inhibits a conversion of acetylCoA to product by Streptococcus pneumoniae or Staphylococcus aureus FabH or a conversion of malonyl-ACP to product by Streptococcus pneumoniae or Staphylococcus aureus FabH.

In any of the methods herein comprising a bacteria, it is preferred that said bacteria is selected from the group consisting of: a member of the genus Staphylococcus, Staphylococcus aureus, a member of the genus Streptococcus, and Streptococcus pneumoniae.

In accordance with another aspect of the invention, there is provided the use of a polynucleotide of the invention for therapeutic or prophylactic purposes, in particular genetic immunization. Among the particularly preferred embodiments of the invention are naturally occurring allelic variants of FabH and polypeptides encoded thereby.

Another aspect of the invention there are provided polypeptides of Staphylococcus aureus referred to herein as FabH as well as biologically, diagnostically, prophylactically, clinically or antibacterially useful variants thereof, and compositions comprising the same.

In accordance with yet another aspect of the invention, there are provided inhibitors to such polypeptides, useful as antibacterial agents, including, for example, antibodies.

In accordance with certain preferred embodiments of the invention, there are provided products, compositions and methods for assessing FabH expression, treating disease, assaying genetic variation, and administering a FabH polypeptide or polynucleotide to an organism to raise an immunological response against a bacteria, especially a Staphylococcus aureus or Streptococcus pneumoniae bacteria.

In certain preferred embodiments of the invention there are provided antibodies against FabH polypeptides.

In other embodiments of the invention there are provided methods for identifying compounds which bind to or otherwise interact with and inhibit or activate an activity of a polypeptide or polynucleotide of the invention comprising: contacting a polypeptide or polynucleotide of the invention with a compound to be screened under conditions to permit binding to or other interaction between the compound and the polypeptide or polynucleotide to assess the binding to or other interaction with the compound, such binding or interaction being associated with a second component capable of providing a detectable signal in response to the binding or interaction of the polypeptide or polynucleotide with the compound; and determining whether the compound binds to or otherwise interacts with and activates or inhibits an activity of the polypeptide or polynucleotide by detecting the presence or absence of a signal generated from the binding or interaction of the compound with the polypeptide or polynucleotide.

In accordance with yet another aspect of the invention, there are provided FabH agonists and antagonists, preferably bacteriostatic or bacteriocidal agonists and antagonists.

In a further aspect of the invention there are provided compositions comprising a FabH polynucleotide, FabH polypeptide or agonist or antagonists thereof for administration to a cell or to a multicellular organism.

Various changes and modifications within the spirit and scope of the disclosed invention will become readily apparent to those skilled in the art from reading the following descriptions and from reading the other parts of the present disclosure.

### DESCRIPTION OF THE INVENTION

The invention relates to modulators of the activity of FabH polypeptides and polynucleotides as described in greater detail below, as well as methods for identifying and using such modulators. In particular, the invention relates to antagonists and agonists of polypeptides and polynucleotides of a FabH, especially of Staphylococcus aureus or Streptococcus pneumoniae. The invention relates especially to antagonists and agonists of FabH having the nucleotide and amino acid sequences set out in Table 1 as SEQ ID NO: 1 and SEQ ID NO: 2 respectively, and to antagonists and agonists of polypeptides encoded by FabH nucleotide sequences of the DNA in the deposited strain.

TABLE 1
FabH Polynucleotide and Polypeptide Sequences

(A) Sequences from Staphylococcus aureus FabH polynucleotide sequence.
The start codon is shown in bold and underlined. The stop codon is underlined.

| | |
|---|---|
| 5′-ATGAACGTGGGTATTAAAGGTTTTGGTGCATATGCACCAGAAAAGA | [SEQ ID NO:1] |
| TTATTGACAATGCCTATTTTGAGCAATTTTTAGATACATCTGATGAATGGATTTCTAAGATGACTGGA | |
| ATTAAAGAAAGACATTGGGCAGATGACGATCAAGATACTTCAGATTTAGCATATGAAGCAAGTGTAAA | |
| AGCAATCGCTGACGCTGGTATTCAGCCTGAAGATATAGATATGATAATTGTTGCCACAGCAACTGGAG | |
| ATATGCCATTTCCAACTGTCGCAAATATGTTGCAAGAACGTTTAGGGACGGGCAAAGTTGCCTCTATG | |
| GATCAACTTGCAGCATGTTCTGGATTTATGTATTCAATGATTACAGCTAAACAATATGTTCAATCTGG | |
| AGATTATCATAATATTTTAGTTGTCGGTGCAGATAAATTATCTAAAATAACAGATTTAACTGACCGTT | |
| CTACTGCAGTTCTATTTGGAGATGGTGCAGGTGCGGTTATCATCGGTGAAGTTTCAGAAGGCAGAGGT | |
| ATTATAAGTTATGAAATGGGTTCTGATGGCACTGGTGGTAAACATTTATATTTAGATAAAGATACTGG | |
| TAAACTGAAAATGAATGGTCGAGAAGTATTTAAATTTGCTGTTAGAATTATGGGTGATGCATCAACAC | |
| GTGTAGTTGAAAAAGCGAATTTAACATCAGATGATATAGATTTATTTATTCCTCATCAAGCTAATATT | |
| AGAATTATGGAATCAGCTAGAGAACGCTTAGGTATTTCAAAAGACAAAATGAGTGTTTCTGTAAATAA | |
| ATATGGAAATACTTCAGCTGCGTCAATACCTTTAAGTATCGATCAAGAATTAAAAAATGGTAAACTCA | |
| AAGATGATGATACAATTGTTCTTGTCGGATTCGGTGGCGGCCTAACTTGGGGCGCAATGACAATAAAA | |
| TGGGGAAAATAG-3′ | |
| (B) Staphylococcus aureus FabH polypeptide sequence deduced from the | |
| polynucleotide sequence (A) in this table. | |
| NH₂-MNVGIKGFGAYAPEKIIDNAYFEQFLDTSDEWISKMTGIKERHWADDD | [SEQ ID NO:2] |
| QDTSDLAYEASVKAIAKAGIQPEDIDMIIVATATGDMPFPTVANMLQERLGTGKVASMDQLAACSGFM | |
| YSMITAKQYVQSGDYHNILVVGADKLSKITDLTDRSTAVLFGDGAGAVIIGEVSEGRGIISYEMGSDG | |
| TGGKHLYLDKDTGKLKMNGREVFKFAVRIMGDASTRVVEKANLTSDDIDLFIPHQANIRIMESARERL | |
| GISKDKMSVSVNKYGNTSAASIPLSIDQELKNGKLKDDDTIVLVGFGGGLTWGAMTIKWGKK-COOH | |

(C) Sequences from Sreptococcus pneumoniae FabH polynucleotide sequence.
The start codon is shown in bold and underlined. The stop codon is underlined.

| | | |
|---|---|---|
| 1 | ATGGCTTTTG CAAAAATAAG TCAGGTTGCT CATTATGTGC CAGAGCAAGT | [SEQ ID NO:3] |
| 51 | GGTTACAAAT CACGACTTGG CTCAGATTAT GGATACCAAT GATGAGTGGA | |
| 101 | TTTCAAGTCG AACGGGAATA CGACAAAGGC ATATTTCAAG AACAGAATCT | |
| 151 | ACCAGTGATT TGGCTACAGA GGTTGCTAAG AAACTGATGG CAAAAGCTGG | |
| 201 | AATAACAGGA AAAGAACTGG ATTTTATCAT CCTAGCTACC ATTACTCCAG | |
| 251 | ATTCGATGAT GCCCTCTACA GCTGCTCGTG TTCAAGCTAA TATTGGCGCT | |
| 301 | AATAAAGCCT TTGCTTTTGA CTTAACCGCG GCTTGCAGTG GATTTGTATT | |
| 351 | TGCTCTTTCA ACTGCTGAAA AGTTTATCGC TTCTGGTCGC TTTCAAAAAG | |
| 401 | GCTTGGTGAT TGGTAGTGAA ACCCTCTCTA AGGCAGTCGA TTGGTCGGAT | |
| 451 | CGATCAACAG CTGTGTTGTT TGGAGATGGT GCTGGTGGTG TCTTGTTAGA | |
| 501 | AGCTAGCGAG CAAGAGCATT TCTTAGCTGA GAGTCTTAAT AGCGATGGAA | |
| 551 | GTCGCAGCGA GTGTTTAACT TATGGGCATT CAGGTTTGCA TTCTCCATTT | |
| 601 | TCAGATCAAG AAAGTGCAGA TTCGTTTTTG AAGATGGATG GACGCACAGT | |
| 651 | CTTTGATTTT GCCATTCGAG ATGTAGCCAA GTCTATCAAG CAGACTATTG | |
| 701 | ATGAATCTCC TATAGAGGTG ACAGACTTGG ATTATCTGCT ACTTCATCAA | |
| 751 | GCCAATGACC GTATTTTGGA TAAGATGGCT AGAAAAATTG GTGTTGACCG | |
| 801 | AGCCAAACTT CCAGCCAATA TGATGGAATA TGGCAATACC AGTGCAGCCA | |
| 851 | GTATCCCGAT TTTACTTTCA GAGTGTGTAG AACAAGGTCT CATCCCTTTA | |
| 901 | GATGGTAGCC AGACTGTTCT TCTATCAGGC TTCGGTGGAG GCTTGACCTG | |
| 951 | GGGCACGCTC ATTCTTACAA TTTAG | |

(D) Streptococcus pneumoniae FabH polypeptide sequence deduced from the
polynucleotide (C) sequence in this table.

| | | |
|---|---|---|
| 1 | NH₂-MAFAKISQVA HYVPEQVVTN HDLAQIMDTN DEWISSRTGI RQRHISRTES | [SEQ ID NO:4] |
| 51 | TSDLATEVAK KLMAKAGITG KELDFIILAT ITPDSMMPST AARVQANIGA | |
| 101 | NKAFAFDLTA ACSGFVFALS TAEKFIASGR FQKGLVIGSE TLSKAVDWSD | |
| 151 | RSTAVLFGDG AGGVLLEASE QEHFLAESLN SDGSRSECLT YGHSGLHSPF | |
| 201 | SDQESADSFL KMDGRTVFDF AIRDVAKSIK QTIDESPIEV TDLDYLLLHQ | |
| 251 | ANDRILDKMA RKIGVDRAKL PANMMEYGNT SAASIPILLS ECVEQGLIPL | |
| 301 | DGSQTVLLSG FGGGLTWGTL ILTI-COOH | |

Deposited Materials

A deposit containing a Staphylococcus aureus WCUH 29 strain has been deposited with the National Collections of Industrial and Marine Bacteria Ltd. (herein “NCIMB”), 23 St. Machar Drive, Aberdeen AB2 IRY, Scotland on Sep. 11, 1995 and assigned NCIMB Deposit No. 40771, and referred to as Staphylococcus aureus WCUH29 on deposit. The Staphylococcus aureus strain deposit is referred to herein as “the deposited strain” or as “the DNA of the deposited strain.”

A deposit containing a Streptococcus pneumoniae bacterial strain has been deposited with the National Collections of Industrial and Marine Bacteria Ltd. (NCIMB), 23 St. Machar Drive, Aberdeen AB2 IRY, Scotland on Apr. 11, 1996 and assigned NCIMB Deposit No. 40794. The Streptococcus pneumoniae bacterial strain deposit is referred to herein as “the deposited bacterial strain” or as “the DNA of the deposited bacterial strain.”

The deposited material is a bacterial strain that contains the full length FabH DNA, referred to as “NCIMB 40794” upon deposit.

Each deposited strain contains a full length FabH gene. The sequence of the polynucleotides contained in a deposited strain, as well as the amino acid sequence of a polypeptide encoded thereby, are controlling in the event of any conflict with any description of sequences herein.

The deposit of the deposited strain has been made under the terms of the Budapest Treaty on the International Recognition of the Deposit of Micro-organisms for Purposes of Patent Procedure. The strain will be irrevocably and without restriction or condition released to the public upon the issuance of a patent. The deposited strain is provided merely as convenience to those of skill in the art and is not an admission that a deposit is required for enablement, such as that required under 35 U.S.C. §112.

A license may be required to make, use or sell the deposited strain, and compounds derived therefrom, and no such license is hereby granted.

Polypeptides

The polypeptides of the invention include a polypeptide of Table 1 [SEQ ID NO:2 OR 4] (in particular the mature polypeptide) as well as polypeptides and fragments, particularly those which have the biological activity of FabH, and also those which have at least 70% identity to a polypeptide of Table 1 [SEQ ID NO: 1 OR 3]or the relevant portion, preferably at least 80% identity to a polypeptide of Table 1 [SEQ ID NO:2 OR 4] and more preferably at least 40%, 50%, 60%, 70%, 80% or 90% similarity (more preferably at least 40%, 50%, 60%, 70%, 80% or 90% identity) to a polypeptide of Table I [SEQ ID NO:2 OR 4] and still more preferably at least 95% similarity (still more preferably at least 95% identity) to a polypeptide of Table I [SEQ ID NO:2 OR 4] and also include portions of such polypeptides with such portion of the polypeptide generally containing at least 30 amino acids and more preferably at least 50 amino acids.

The invention also includes polypeptides of the formula:

X—(R₁)ₘ—(R₂)—(R₃)ₙ—Y

wherein, at the amino terminus, X is hydrogen, and at the carboxyl terminus, Y is hydrogen or a metal, R₁ and R₃ are any amino acid residue, m is an integer between 1 and 1000 or zero, n is an integer between 1 and 1000 or zero, and R₂ is an amino acid sequence of the invention, particularly an amino acid sequence selected from Table 1. In the formula above R₂ is oriented so that its amino terminal residue is at the left, bound to RI, and its carboxy terminal residue is at the right, bound to R₃. Any stretch of amino acid residues denoted by either R group, where m and/or n is greater than 1, may be either a heteropolymer or a homopolymer, preferably a heteropolymer.

A fragment is a variant polypeptide having an amino acid sequence that entirely is the same as part but not all of the amino acid sequence of the aforementioned polypeptides. As with FabH polypeptides fragments may be “free-standing,” or comprised within a larger polypeptide of which they form a part or region, most preferably as a single continuous region, a single larger polypeptide.

Preferred fragments include, for example, truncation polypeptides having a portion of an amino acid sequence of Table 1 [SEQ ID NO:2 OR 4], or of variants thereof, such as a continuous series of residues that includes the amino terminus, or a continuous series of residues that includes the carboxyl terminus. Degradation forms of the polypeptides of the invention in a host cell, particularly a Staphylococcus aureus or Streptococcus pneumoniae, are also preferred. Further preferred are fragments characterized by structural or functional attributes such as fragments that comprise alpha-helix and alpha-helix forming regions, beta-sheet and beta-sheet-forming regions, turn and tum-forming regions, coil and coil-forming regions, hydrophilic regions, hydrophobic regions, alpha amphipathic regions, beta amphipathic regions, flexible regions, surface-forming regions, substrate binding region, and high antigenic index regions.

Also preferred are biologically active fragments which are those fragments that mediate activities of FabH, including those with a similar activity or an improved activity, or with a decreased undesirable activity. Also included are those fragments that are antigenic or immunogenic in an animal, especially in a human. Particularly preferred are fragments comprising receptors or domains of enzymes that confer a function essential for viability of Staphylococcus aureus or Streptococcus pneumoniae or the ability to initiate, or maintain cause disease in an individual, particularly a human.

Variants that are fragments of the polypeptides of the invention may be employed for producing the corresponding full-length polypeptide by peptide synthesis; therefore, these variants may be employed as intermediates for producing the full-length polypeptides of the invention.

Polynucleotides

Another aspect of the invention relates to isolated polynucleotides, including the full length gene, that encode the FabH polypeptide having a deduced amino acid sequence of Table 1 [SEQ ID NO:2 OR 4] and polynucleotides closely related thereto and variants thereof.

Using the information provided herein, such as a polynucleotide sequence set out in Table I [SEQ ID NO: 1 OR 3], a polynucleotide of the invention encoding FabH polypeptide may be obtained using standard cloning and screening methods, such as those for cloning and sequencing chromosomal DNA fragments from bacteria using Staphylococcus aureus WCUH 29 cells as starting material, followed by obtaining a full length clone. For example, to obtain a polynucleotide sequence of the invention, such as a sequence given in Table I [SEQ ID NO:1 OR 3], typically a library of clones of chromosomal DNA of Staphylococcus aureus WCUH 29 in E. coli or some other suitable host is probed with a radiolabeled oligonucleotide, preferably a 17-mer or longer, derived from a partial sequence. Clones carrying DNA identical to that of the probe can then be distinguished using stringent conditions. By sequencing the individual clones thus identified with sequencing primers designed from the original sequence it is then possible to extend the sequence in both directions to determine the full gene sequence. Conveniently, such sequencing is performed using denatured double stranded DNA prepared from a plasmid clone. Suitable techniques are described by Maniatis, T., Fritsch, E. F. and Sambrook et al., MOLECULAR CLONING, A IABORATORY MANUAL, 2nd Ed.; Cold Spring Harbor Laboratory Press, Cold Spring Harbor, N.Y. (1989). (see in particular Screening By Hybridization 1.90 and Sequencing Denatured Double-Stranded DNA Templates 13.70). Illustrative of the invention, the polynucleotide set out in Table I [SEQ ID NO: 1 OR 3] was discovered in a DNA library derived from Staphylococcus aureus WCUH 29.

The DNA sequence set out in Table I [SEQ ID NO: 1 OR 3] contains an open reading frame encoding a protein having about the number of amino acid residues set forth in Table I [SEQ ID NO:2 OR 4] with a deduced molecular weight that can be calculated using amino acid residue molecular weight values well known in the art. The polynucleotide of SEQ ID NO: 1, between nucleotide number 1 and the stop codon which begins at the third nucleotide from the 3′-end of SEQ ID NO: 1 OR 3, encodes the polypeptide of SEQ ID NO:2 OR 4.

FabH of the invention is structurally related to other proteins of the Fab family, as shown by the results of sequencing the DNA encoding FabH of the deposited strain.

The invention provides a polynucleotide sequence identical over its entire length to a coding sequence in Table I [SEQ ID NO:1 OR 3]. Also provided by the invention is the coding sequence for the mature polypeptide or a fragment thereof, by itself as well as the coding sequence for the mature polypeptide or a fragment in reading frame with other coding sequence, such as those encoding a leader or secretory sequence, a pre-, or pro- or prepro-protein sequence. The polynucleotide may also contain non-coding sequences, including for example, but not limited to non-coding 5′ and 3′ sequences, such as the transcribed, non-translated sequences, termination signals, ribosome binding sites, sequences that stabilize mRNA, introns, polyadenylation signals, and additional coding sequence which encode additional amino acids. For example, a marker sequence that facilitates purification of the fused polypeptide can be encoded. In certain embodiments of the invention, the marker sequence is a hexa-histidine peptide, as provided in the pQE vector (Qiagen, Inc.) and described in Gentz et al., Proc. Natl. Acad. Sci., USA 86: 821-824 (1989), or an HA tag (Wilson et al., Cell 37: 767 (1984). Polynucleotides of the invention also include, but are not limited to, polynucleotides comprising a structural gene and its naturally associated sequences that control gene expression.

The invention also includes polynucleotides of the formula:

X—(R₁)ₘ—(R₂)—(R₃)ₙ—Y

wherein, at the 5′ end of the molecule, X is hydrogen, and at the 3′ end of the molecule, Y is hydrogen or a metal, R₁ and R₃ is any nucleic acid residue, m is an integer between 1 and 3000 or zero, n is an integer between 1 and 3000 or zero, and R₂ is a nucleic acid sequence of the invention, particularly a nucleic acid sequence selected from Table 1. In the polynucleotide formula above R₂ is oriented so that its 5′ end residue is at the left, bound to R₁, and its 3′ end residue is at the right, bound to R₃. Any stretch of nucleic acid residues denoted by either R group, where m and/or n is greater than 1, may be either a heteropolymer or a homopolymer, preferably a heteropolymer. In a preferred embodiment m and/or n is an integer between 1 and 1000.

It is most preferred that the polynucleotides of the inventions are derived from Staphylococcus aureus or Streptococcus pneumoniae, however, they may preferably be obtained from organisms of the same taxonomic genus. They may also be obtained, for example, from organisims of the same taxonomic family or order.

The term “polynucleotide encoding a polypeptide” as used herein encompasses polynucleotides that include a sequence encoding a polypeptide of the invention, particularly a bacterial polypeptide and more particularly a polypeptide of the Staphylococcus aureus or Streptococcus pneumoniae FabH having an amino acid sequence set out in Table I [SEQ ID NO:2 OR 4]. The terrn also encompasses polynucleotides that include a single continuous region or discontinuous regions encoding the polypeptide (for example, interrupted by integrated phage or an insertion sequence or editing) together with additional regions, that also may contain coding and/or non-oding sequences.

The invention further relates to variants of the polynucleotides described herein that encode for variants of the polypeptide having a deduced amino acid sequence of Table 1 [SEQ ID NO:2 OR 4]. Variants that are fragments of the polynucleotides of the invention may be used to synthesize full-length polynucleotides of the invention.

Further particularly preferred embodiments are polynucleotides encoding FabH variants, that have the amino acid sequence of FabH polypeptide of Table 1 [SEQ ID NO:2 OR 4] in which several, a few, 5 to 10, 1 to 5, 1 to 3, 2, 1 or no amino acid residues are substituted, deleted or added, in any combination. Especially preferred among these are silent substitutions, additions and deletions, that do not alter the properties and activities of FabH.

Further preferred embodiments of the invention are polynucleotides that are at least 40%, 50%, 60%, or 70% identical over their entire length to a polynucleotide encoding FabH polypeptide having an amino acid sequence set out in Table 1 [SEQ ID NO:2 OR 4], and polynucleotides that are complementary to such polynucleotides. Alternatively, most highly preferred are polynucleotides that comprise a region that is at least 80% identical over its entire length to a polynucleotide encoding FabH polypeptide of the deposited strain and polynucleotides complementary thereto. In this regard, polynucleotides at least 40%, 50%, 60%, 70%, 80% or 90% identical over their entire length to the same are particularly preferred, and among these particularly preferred polynucleotides, those with at least 95% are especially preferred. Furthermore, those with at least 97% are highly preferred among those with at least 95%, and among these those with at least 98% and at least 99% are particularly highly preferred, with at least 99% being the more preferred.

Preferred embodiments are polynucleotides that encode polypeptides that retain substantially the same biological function or activity as the mature polypeptide encoded by a DNA of Table 1 [SEQ ID NO: 1 OR 3].

The invention further relates to polynucleotides that hybridize to the herein above-described sequences. In this regard, the invention especially relates to polynucleotides that hybridize under stringent conditions to the herein above-described polynucleotides. As herein used, the terms “stringent conditions” and “stringent hybridization conditions” mean hybridization will occur only if there is at least 95% and preferably at least 97% identity between the sequences. An example of stringent hybridization conditions is overnight incubation at 42° C. in a solution comprising: 50% fornamide, 5× SSC (150 mM NaCl, 15 mM trisodium citrate), 50 mM sodium phosphate (pH7.6), 5× Denhardt's solution, 10% dextran sulfate, and 20 micrograms/ml denatured, sheared salmon sperm DNA, followed by washing the hybridization support in 0.1× SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein.

The invention also provides a polynucleotide consisting essentially of a polynucleotide sequence obtainable by screening an appropriate library containing the complete gene for a polynucleotide sequence set forth in SEQ ID NO: 1 OR 3 under stringent hybridization conditions with a probe having the sequence of said polynucleotide sequence set forth in SEQ ID NO: 1 OR 3 or a fragment thereof; and isolating said DNA sequence. Fragments useful for obtaining such a polynucleotide include, for example, probes and primers described elsewhere herein.

As discussed additionally herein regarding polynucleotide assays of the invention, for instance, polynucleotides of the invention as discussed above, may be used as a hybridization probe for RNA, cDNA and genomic DNA to isolate full-length cDNAs and genomic clones encoding FabH and to isolate cDNA and genomic clones of other genes that have a high sequence similarity to the FabH gene. Such probes generally will comprise at least 15 bases. Preferably, such probes will have at least 30 bases and may have at least 50 bases. Particularly preferred probes will have at least 30 bases and will have 50 bases or less.

For example, the coding region of the FabH gene may be isolated by screening using a DNA sequence provided in Table 1 [SEQ ID NO: 1 or 3] to synthesize an oligonucleotide probe. A labeled oligonucleotide having a sequence complementary to that of a gene of the invention is then used to screen a library of cDNA, genomic DNA or mRNA to determine which members of the library the probe hybridizes to.

The polynucleotides and polypeptides of the invention may be employed, for example, as research reagents and materials for discovery of treatments of and diagnostics for disease, particularly human disease, as further discussed herein relating to polynucleotide assays.

Polynucleotides of the invention that are oligonucleotides derived from the sequences of Table 1 [SEQ ID NOS:1, 2, 3 or 4] may be used in the processes herein as described, but preferably for PCR, to determine whether or not the polynucleotides identified herein in whole or in part are transcribed in bacteria in infected tissue. It is recognized that such sequences will also have utility in diagnosis of the stage of infection and type of infection the pathogen has attained.

The invention also provides polynucleotides that may encode a polypeptide that is the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from the mature protein by cellular enzymes.

A precursor protein, having the mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

In sum, a polynucleotide of the invention may encode a mature protein, a mature protein plus a leader sequence (which may be referred to as a preprotein), a precursor of a mature protein having one or more prosequences that are not the leader sequences of a preprotein, or a preproprotein, which is a precursor to a proprotein, having a leader sequence and one or more prosequences, which generally are removed during processing steps that produce active and mature forms of the polypeptide.

Vectors, Host Cells, Expression

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention.

For recombinant production, host cells can be genetically engineered to incorporate expression systems or portions thereof or polynucleotides of the invention. Introduction of a polynucleotide into the host cell can be effected by methods described in many standard laboratory manuals, such as Davis et al., BASIC METHODS IN MOLECULAR BIOLOGY, (1986) and Sambrook et al., MOLECULAR CLONING: A LABORATORYMANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as streptococci, staphylococci, enterococci E. coli, streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A great variety of expression systems can be used to produce the polypeptides of the invention. Such vectors include, among others, chromosomal, episomal and virus-derived vectors, e-g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding protein may be employed to regenerate active conformation when the polypeptide is denatured during isolation and or purification.

Antibodies

The polypeptides of the invention or variants thereof, or cells expressing them can be used as an immunogen to produce antibodies immunospecific for such polypeptides. “Antibodies” as used herein includes monoclonal and polyclonal antibodies, chimeric, single chain, sirnianized antibodies and humanized antibodies, as well as Fab fragments, including the products of an Fab immunolglobulin expression library.

Antibodies generated against the polypeptides of the invention can be obtained by admninistering the polypeptides or epitope-bearing fragments, analogues or cells to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in MONOCLONAL ANTIBODIES AND CANCER THERAPY, Alan R. Liss, Inc. (1985).

Techniques for the production of single chain antibodies (U.S. Pat. No. 4,946,778) can be adapted to produce single chain antibodies to polypeptides of this invention. Also, transgenic mice, or other organisms such as other mammals, may be used to express humanized antibodies.

Alternatively phage display technology may be utilized to select antibody genes with binding activities towards the polypeptide either from repertoires of PCR amplified v-genes of lymphocytes from humans screened for possessing anti-FabH or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

If two antigen binding domains are present each domain may be directed against a different epitope - termed bispecific′ antibodies.

The above-described antibodies may be employed to isolate or to identify clones expressing the polypeptides to purify the polypeptides by affinity chromatography.

Thus, among others, antibodies against FabH- polypeptide may be employed to treat infections, particularly bacterial infections.

Polypeptide variants include antigenically, epitopically or immunologically equivalent variants that form a particular aspect of this invention. The term “antigenically equivalent derivative” as used herein encompasses a polypeptide or its equivalent which will be specifically recognized by certain antibodies which, when raised to the protein or polypeptide according to the invention, interfere with the immediate physical interaction between pathogen and mammalian host. The term “immunologically equivalent derivative” as used herein encompasses a peptide or its equivalent which when used in a suitable formulation to raise antibodies in a vertebrate, the antibodies act to interfere with the immediate physical interaction between pathogen and mammalian host.

The polypeptide, such as an antigenically or immunologically equivalent derivative or a fusion protein thereof is used as an antigen to immunize a mouse or other animal such as a rat or chicken. The fusion protein may provide stability to the polypeptide. The antigen may be associated, for example by conjugation, with an immunogenic carrier protein for example bovine serum albumin (BSA) or keyhole limpet haemocyanin (KLH). Alternatively a multiple antigenic peptide comprising multiple copies of the protein or polypeptide, or an antigenically or immunologically equivalent polypeptide thereof may be sufficiently antigenic to improve immunogenicity so as to obviate the use of a carrier.

Preferably, the antibody or variant thereof is modified to make it less immunogenic in the individual. For example, if the individual is human the antibody may most preferably be “humanized”; where the complimentarily determining region(s) of the hybridoma-derived antibody has been transplanted into a human monoclonal antibody , for example as described in Jones, P. et al. (1986), Nature 321, 522-525 or Tempest et al., (1991) Biotechnology 9, 266-273.

The use of a polynucleotide of the invention in genetic immunization will preferably employ a suitable delivery method such as direct injection of plasmid DNA into muscles (Wolff et al., Hum Mol Genet 1992, 1:363, Manthorpe et al., Hum. Gene Ther. 1963:4, 419), delivery of DNA complexed with specific protein carriers (Wu et al., J Biol Chem. 1989: 264,16985), coprecipitation of DNA with calcium phosphate (Benvenista & Reshef, PNAS USA, 1986:83,9551), encapsulation of DNA in various forms of liposomes (Kaneda et al., Science 1989:243,375), particle bombardment (Tang et al., Nature 1992, 356:152, Eisenbraun et al., DNA Cell Biol 1993, 12:791) and in vivo infection using cloned retroviral vectors (Seeger et al., PNAS USA 1984:81,5849).

Mechanisms of Action of FabH: Antagonists and Agonists and Methods of Use

Polypeptides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991).

The invention also provides a method of screening compounds to identify or select those which enhance (agonist) or block (antagonist) the action of the ping-pong reaction of FabH polypeptides, particularly those compounds that are bacteriostatic and/or bacteriocidal. The method of screening may involve high-throughput techniques. For example, to screen for agonists or antagonists, a synthetic reaction mix, a cellular compartment, such as a membrane, cell envelope or cell wall, or a preparation of any thereof, comprising FabH polypeptide and a labeled substrate or ligand of such polypeptide is incubated in the absence or the presence of a candidate molecule that may be a FabH agonist or antagonist. The ability of the candidate molecule to agonize or antagonize the FabH polypeptide is reflected in increased or decreased binding of the labeled ligand increased or decreased production of product from such substrate. Molecules that bind gratuitously, i.e., without inducing the effects of FabH polypeptide are most likely to be good antagonists. Molecules that bind well and increase the rate of product production from substrate are agonists. Detection of the rate or level of production of product from substrate may be enhanced by using a reporter system. Reporter systems that may be useful in this regard include but are not limited to radioactively labeled substrate or colorimetric labeled substrate converted into product, a reporter gene that is responsive to changes in FabH polynucleotide or polypeptide activity, and binding assays known in the art.

Another example of an assay for the identification or selection FabH antagonists or agonists is a competitive assay that combines FabH and a potential antagonist or agonist (herein “candidate compound,” which can be any chemical element, compound or composition) with FabH-binding molecules, recombinant FabH binding molecules, natural substrates or ligands, or substrate (e.g., acetyl-CoA, malonyl-ACP or derivatives thereof) or ligand rimretics, under appropriate conditions for a competitive inhibition assay. FabH can be labeled, such as by radioactivity or a colorimetric compound, such that the number of FabH molecules bound to a binding molecule or converted to product can be determined accurately to assess the effectiveness of the potential antagonist.

Preferred methods of screening comprise the steps of adding a candidate compound to a reaction mixture comprising FabH, particularly S. aureus or S. pneumoniae FabH and detecting modulation of a conversion of acetyl-CoA (the first substrate in the ping-pong reaction) to product and or a conversion of malonyl-ACP (the second substrate in the ping-pong reaction) to product. It is most preferred that this modulation be antagonism or agonism of either or both of the ping-pong reaction steps.

It is also preferred that any method of screening of the invention be carried out such that modulation of a conversion of acetylCoA, or a derivative thereof, to product is followed by modulation of a conversion of malonyl-ACP, or a derivative thereof, to product. It is most preferred that this modulation be antagonism or agonism. Preferred compounds modulate the activity of both reactions.

Based on the teachings herein the skilled artisan could configure a variety of screens for compounds that modulate the activity of the FabH ping-pong reaction.

The invention also provides compounds, particularly small molecule compounds, that modulate an activity or expression of a polypeptide of the invention, but particularly, a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4.

The invention further provides a method for the treatment of an individual having need to inhibit FabH polypeptide, such as an individual infected by bacteria, comprising the steps of: administering to the individual a antibacterially effective amount of an antagonist that inhibits, or an agonists that activates, an activity of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4.

Also provided is a method for the treatment of an individual infected with a bacteria, preferably a bacteria of the genera Streptococcus or Stapylococcus, comprising the steps of: administering to the individual a antibacterially effective amount of an antagonist that inhibits, or an agonist that activates, an activity of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4.

Further provided is a method for the treatment of an individual having need to inhibit FabH polypeptide, such as an individual infected by bacteria, comprising the steps of: administering to the individual a antibacterially effective amount of an antagonist that inhibits a conversion of acetylCoA to product or a conversion of malonyl-ACP to product, particularly by modulating the kinetics of a ping-pong reaction.

The invention also provides a method for the treatment of an individual infected with a bacterium having comprising the steps of: administering to the individual a antibacterially effective amount of an antagonist that inhibits a conversion of acetyl-CoA by FabH to product or a conversion of malonyl-ACP by FabH to product particularly by modulating the kinetics of a ping-pong reaction.

Also provided is a method for the treatment of an individual infected by a bacteria comprising the steps of: administering to the individual a antibacterially effective amount of an antagonist that inhibits a conversion of acetyl-CoA to product by Staphylococcus aureus FabH or a conversion of rnalonyl-ACP to product by Staphylococcus aureus FabH.

Still further provided is a method for the treatment of an individual infected by Streptococcus pneumoniae comprising the steps of: administering to the individual a antibacterially effective amount of an antagonist that inhibits a conversion of acetyl-CoA to product by Streptococcus pneumoniae FabH or a conversion of malonyl-ACP to product by Streptococcus pneumoniae FabH particularly by modulating the kinetics of a ping-pong reaction.

Yet another method provides an antagonist that inhibits an activity of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4, wherein said activity is a conversion of acetyl-CoA to product or a conversion of malonyl-ACP to product particularly by modulating the kinetics of a ping-pong reaction.

This invention provides another method for the treatment of an individual having need to inhibit FabH polypeptide comprising the steps of: administering to the individual a antibacterially effective amount of an antagonist that inhibits an activity of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4, wherein said activity is a conversion of acetyl-CoA to product or a conversion of malonyl-ACP to product particularly by modulating the kinetics of a ping-pong reaction.

Also provided by the invention is a method for the treatment of an individual infected with a bacterium comprising the steps of: administering to the individual a antibacterially effective amount of an antagonist that inhibits an activity of a polypeptide selected from the group consisting of: a polypeptide comprising an amino acid sequence which is at least 40%, 50%, 60%, 70%, 80% or 90% identical to the amino acid sequence of SEQ ID NO:2 OR 4, and a polypeptide comprising an amino acid sequence as set forth in SEQ ID NO:2 OR 4 wherein said activity is a conversion of acetyl-CoA to product or a conversion of malonyl-ACP to product particularly by modulating the kinetics of a ping-pong reaction.

A method for inhibiting a FabH polypeptide comprising the steps of: contacting a composition comprising said polypeptide with an amount effective amount of an antagonist that inhibits a conversion of acetyl-CoA to product or a conversion of malonyl-ACP to product, particularly by modulating the kinetics of a ping-pong reaction, is also provided by the invention.

A method for inhibiting a conversion of acetyl-CoA to product or a conversion of malonyl-ACP to product comprising the steps of: contacting a composition comprising bacteria with a compound that inhibits a conversion of acetyl-CoA to product or a conversion of malonyl-ACP to product for an effective time to cause killing or slowing of growth of said bacteria, particularly by modulating the kinetics of a ping-pong reaction, is also provided herein.

The invention also provides a method for inhibiting a growth of bacteria comprising the steps of: contacting a composition comprising bacteria with an antibacterially effective amount of an antagonist that inhibits a conversion of acetyl-CoA to product by Streptococcus pneumoniae or Staphylococcus aureus FabH or a conversion of malonyl-ACP to product by Streptococcus pneumoniae or Staphylococcus aureus FabH particularly by modulating the kinetics of a ping-pong reaction.

A method is also provided by the invention for inhibiting a FabH polypeptide comprising the steps of: contacting a composition comprising bacteria with an antibacterially effective amount of an antagonist that inhibits a conversion of acetyl-CoA to product by Streptococcus pneumoniae or Staphylococcus aureus FabH or a conversion of malonyl-ACP to product by Streptococcus pneumoniae or Staphylococcus aureus FabH particularly by modulating the kinetics of a ping-pong reaction.

In any of the methods herein comprising a bacteria, it is preferred that said bacteria is selected from the group consisting of: a member of the genus Staphylococcus, Staphylococcus aureus, a member of the genus Streptococcus, and Streptococcus pneumoniae.

It is preferred that in the methods of the invention the product of the first step of the ping-pong reaction is acetyl-FabH.

Preferred agonists of the invention enhance the level of condensation of acetyl-CoA with malonyl-ACP, particularly to increase the initiation of fatty acid biosynthesis in dissociated, type II, fatty acid synthase systems typified by bacteria of the genera Staphylococcus, Streptococcus or Escherichia.

Preferred antagonists of the invention decrease the level of condensation of acetyl-CoA with malonyl-ACP, particularly to decrease the initiation of fatty acid biosynthesis in dissociated, type II, fatty acid synthase systems as set forth herein.

A preferred embodiment of the methods of the invention provide for detection of modulation of a FabH activity by kinetic analysis whereby one can show modulation of the ping-pong mechanism. A more preferred method to detect modulation of a FabH activity is by measuring or otherwise detecting the acetyl-FabH product, and a modulation of the level of product after addition of a candidate compound as compared to a control reaction with no candidate compound. Another preferred method to detect modulation of an a FabH activity is by measuring an apparent Km after addition of a candidate compound as compared to a control reaction with no candidate compound. In still another preferred embodiment, modulation of the Km to between 0 and 4 micromolar for malonyl-ACP, an/or modulation of the Km to between 0 and 25 micromolar for acetyl-CoA demonstrates that the candidate compound is an agonist. In yet another preferred embodiment, modulation of the Km to between 7 and 100 or more micromolar for malonyl-ACP, an/or modulation of the Km to between 40 and 100 or more micromolar for acetyl-CoA demonstrates that the candidate compound is an antagonist. In a more preferred embodiment, modulation of the Km to between 0 and 1 micromolar for malonyl-ACP, an/or modulation of the Km to between 0 and 10 micromolar for acetyl-CoA demonstrates that the candidate compound is a preferred agonist. In yet another more preferred embodiment, modulation of the Km to between 40 and 200 micromolar for malonyl-ACP, an/or modulation of the Km to between 100 and 200 micromolar for acetyl-CoA demonstrates that the candidate compound is a preferred antagonist.

In accordance with another aspect of the invention, there is provided the use of a polynucleotide of the invention for therapeutic or prophylactic purposes, in particular genetic immunization. Among the particularly preferred embodiments of the invention are naturally occurring allelic variants of FabH and polypeptides encoded thereby.

Another aspect of the invention there are provided polypeptides of Staphylococcus aureus referred to herein as FabH as well as biologically, diagnostically, prophylactically, clinically or antibacterially useful variants thereof, and compositions comprising the same.

In accordance with yet another aspect of the invention, there are provided inhibitors to such polypeptides, useful as antibacterial agents, including, for example, antibodies.

In accordance with certain preferred embodiments of the invention, there are provided products, compositions and methods for assessing FabH expression, treating disease, assaying genetic variation, and administering a FabH polypeptide or polynucleotide to an organism to raise an immunological response against a bacteria, especially a Staphylococcus aureus or Streptococcus pneumoniae bacteria.

In certain preferred embodiments of the invention there are provided antibodies against FabH polypeptides.

In other embodiments of the invention there are provided methods for identifying compounds which bind to or otherwise interact with and inhibit or activate an activity of a polypeptide or polynucleotide of the invention comprising: contacting a polypeptide or polynucleotide of the invention with a compound to be screened tinder conditions to permit binding to or other interaction between the compound and the polypeptide or polynucleotide to assess the binding to or other interaction with the compound, such binding or interaction being associated with a second component capable of providing a detectable signal in response to the binding or interaction of the polypeptide or polynucleotide with the compound; and determining whether the compound binds to or otherwise interacts with and activates or inhibits an activity of the polypeptide or polynucleotide by detecting the presence or absence of a signal generated from the binding or interaction of the compound with the polypeptide or polynucleotide.

In accordance with yet another aspect of the invention, there are provided FabH agonists and antagonists, preferably bacteriostatic or bacteriocidal agonists and antagonists.

In a further aspect of the invention there are provided compositions comprising a FabH polynucleotide, FabH polypeptide or agonist or antagonists thereof for administration to a cell or to a multicellular organism.

Various changes and modifications within the spirit and scope of the disclosed invention will become readily apparent to those skilled in the art from reading the following descriptions and from reading the other parts of the present disclosure.

Potential antagonists include small organic molecules, peptides, polypeptides and antibodies that bind to a polynucleotide or polypeptide of the invention and thereby inhibit or extinguish its activity. Potential antagonists also may be small organic molecules, a peptide, a polypeptide such as a closely related protein or antibody that binds the same sites on a binding molecule, such as a binding molecule, without inducing FabH-induced activities, thereby preventing the action of FabH by excluding FabH from binding.

Potential antagonists include a small molecule that binds to and occupies the binding site of the polypeptide thereby preventing binding to cellular binding molecules, such that normal biological activity is prevented. Examples of small molecules include but are not limited to small organic molecules, peptides or peptide-like molecules. Other potential antagonists include antisense molecules (see Okano, J. Neurochem. 56: 560 (1991); OLIGODEOXYNUCLEOTIDES AS ANTISENSE INHIBITORS OF GENE EXPRESSION, CRC Press, Boca Raton, Fla. (1988), for a description of these molecules). Preferred potential antagonists include compounds related to and variants of FabH.

Small organic molecules of the invention preferably have a molecular weight below 2,000 daltons, more preferably between 300 and 1,000 daltons, and most preferably between 400 and 700 daltons. An embodiment of the invention provides small molecules are those which exclude cerulenin or derivatives thereof and/or thiolactomycin or derivatives thereof. Another embodiment of the invention provides small molecules which are thiolactomycin and/or derivatives thereof. Still another embodiment provides compounds that were not published prior to the filing date of this application.

Each of the DNA sequences provided herein may be used in the discovery and development of antibacterial compounds. The encoded protein, upon expression, can be used as a target for the screening of antibacterial drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest.

The antagonists and agonists of the invention may be employed, for instance, to inhibit and treat diseases.

Helicobacter pylori (herein H. pylori) bacteria infect the stomachs of over one-third of the world's population causing stomach cancer, ulcers, and gastritis (International Agency for Research on Cancer (1994) Schistosomes, Liver Flukes and Helicobacter Pylori (International Agency for Research on Cancer, Lyon, France; http://www.uicc.ch/ecp/ecp2904.htm). Moreover, the international Agency for Research on Cancer recently recognized a cause-and-effect relationship between H. pylori and gastric adenocarcinoma, classifying the bacterium as a Group I (definite) carcinogen. Preferred antimicrobial compounds of the invention (agonists and antagonists of FabH) found using screens provided by the invention, particularly broad-spectrum antibiotics, should be useful in the treatment of H. pylori infection. Such treatment should decrease the advent of H. pylori-induced cancers; such as gastrointestinal carcinoma. Such treatment should also cure gastric ulcers and gastritis.

Compositions, Kits and Administration

The invention also relates to compositions comprising the polynucleotide or the polypeptides discussed above or their agonists or antagonists. The polypeptides of the invention may be employed in combination with a non-sterile or sterile carrier or carriers for use with cells, tissues or organisms, such as a pharmaceutical carrier suitable for administration to a subject. Such compositions comprise, for instance, a media additive or a antibacterially effective amount of a polypeptide of the invention and a pharmaceutically acceptable carrier or excipient. Such carriers may include, but are not limited to, saline, buffered saline, dextrose, water, glycerol, ethanol and combinations thereof. The formulation should suit the mode of administration. The invention further relates to diagnostic and pharmaceutical packs and kits comprising one or more containers filled with one or more of the ingredients of the aforementioned compositions of the invention.

Polypeptides and other compounds of the invention may be employed alone or in conjunction with other compounds, such as therapeutic compounds.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic.

Alternatively the composition may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to mammals, and particularly humans, it is expected that the daily dosage level of the active agent will be from 0.01 mg/kg to 10 mg/kg, typically around 1 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

In-dwelling devices include surgical implants, prosthetic devices and catheters, i.e., devices that are introduced to the body of an individual and remain in position for. an extended time. Such devices include, for example, artificial joints, heart valves, pacemakers, vascular grafts, vascular catheters, cerebrospinal fluid shunts, urinary catheters, continuous ambulatory peritoneal dialysis (CAPD) catheters.

The composition of the invention may be administered by injection to achieve a systemic effect against relevant bacteria shortly before insertion of an in-dwelling device. Treatment may be continued after surgery during the in-body time of the device. In addition, the composition could also be used to broaden perioperative cover for any surgical technique to prevent bacterial wound infections, especially Staphylococcus aureus or Streptococcus pneumoniae wound infections.

Many orthopaedic surgeons consider that humans with prosthetic joints should be considered for antibiotic prophylaxis before dental treatment that could produce a bacteremia. Late deep infection is a serious complication sometimes leading to loss of the prosthetic joint and is accompanied by significant morbidity and mortality. It may therefore be possible to extend the use of the active agent as a replacement for prophylactic antibiotics in this situation.

In addition to the therapy described above, the compositions of this invention may be used generally as a wound treatment agent to prevent adhesion of bacteria to matrix proteins exposed in wound tissue and for prophylactic use in dental treatment as an alternative to, or in conjunction with, antibiotic prophylaxis.

Alternatively, the composition of the invention may be used to bathe an indwelling device immediately before insertion. The active agent will preferably be present at a concentration of 1 μg/ml to 10 mg/ml for bathing of wounds or indwelling devices.

A vaccine composition is conveniently in injectable form. Conventional adjuvants may be employed to enhance the immune response. A suitable unit dose for vaccination is 0.5-5 microgram/kg of antigen, and such dose is preferably administered 1-3 times and with an interval of 1-3 weeks. With the indicated dose range, no adverse toxicological effects will be observed with the compounds of the invention which would preclude their administration to suitable individuals.

Each reference disclosed herein is incorporated by reference herein in its entirety. Any patent application to which this application claims priority is also incorporated by reference herein in its entirety.

Glossary

The following definitions are provided to facilitate understanding of certain terms used frequently herein.

“Bodily material(s) means any material derived from an individual or from an organism infecting, infesting or inhabiting an individual, including but not limited to, cells, tissues and waste, such as, bone, blood, serum, cerebrospinal fluid, semen, saliva, muscle, cartilage, organ tissue, skin, urine, stool or autopsy materials.

“Disease(s)” means any disease caused by or related to infection by a bacteria, including, for example, infections of the upper respiratory tract (e.g., otitis media, bacterial tracheitis, acute epiglottitis, thyroiditis), lower respiratory (e.g., empyema, lung abscess), cardiac (e.g., infective endocarditis), gastrointestinal (e.g., secretory diarrhoea, splenic absces, retroperitoneal abscess), CNS (e.g., cerebral abscess), eye (e.g., blepharitis, conjunctivitis, keratitis, endophthalmitis, preseptal and orbital cellulitis, darcryocystitis), kidney and urinary tract (e.g., epididymitis, intrarenal and perinephric absces, toxic shock syndrome), skin (e.g., impetigo, folliculitis, cutaneous abscesses, cellulitis, wound infection, bacterial myositis) bone and joint (e.g., septic arthritis, osteomyelitis).

“Host cell(s)” is a cell that has been introduced (e.g., transformed or transfected) or is capable of introduction (e.g., transformation or transfection) by an exogenous polynucleotide sequence.

“Identity,” as known in the art, is a relationship between two or more polypeptide sequences or two or more polynucleotide sequences, as the case may be, as determined by comparing the sequences. In the art, “identity”0 also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S.F. et al., J. Molec. Biol. 215: 403-410 (1990). The BLAST X program is publicly available from NCBI and other sources (BLAST Manual, Altschul, S., et aL, NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following: Algorithm: Needleman and Wunsch, J. Mol Biol. 48: 443-453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: Algorithm:

Needleman and Wunsch, J. Mol Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (I) and (2) below. (1) Polynucleotide embodiments further include an isolated polynucleotide comprising a polynucleotide sequence having at least a 95, 97 or 100% identity to the reference sequence of SEQ ID NO: 1 OR 3, wherein said polynucleotide sequence may be identical to the reference sequence of SEQ ID NO: 1 OR 3 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NO: 1 OR 3 by the integer defining the percent identity divided by 100 and then subtracting that product from said total number of nucleotides in SEQ ID NO: 1 OR 3, or:

nₙ≦xₙ−(xωy),

wherein nn is the number of nucleotide alterations, xₙ is the total number of nucleotides in SEQ ID NO:1 OR 3, y is 0.95 for 95%, 0.97 for 97% or 1.00 for 100%, and • is the symbol for the multiplication operator, and wherein any non-integer product of xₙ and y is rounded down to the nearest integer prior to subtracting it from xₙ. Alterations of a polynucleotide sequence encoding the polypeptide of SEQ ID NO:2 OR 4 may create nonsense, missense or frameshift mutations in this coding sequence and thereby alter the polypeptide encoded by the polynucleotide following such alterations.

(2) Polypeptide embodiments further include an isolated polypeptide comprising a polypeptide having at least a 95, 97 or 100% identity to a polypeptide reference sequence of SEQ ID NO:2 OR 4, wherein said polypeptide sequence may be identical to the reference sequence of SEQ ID NO:2 OR 4 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:2 OR 4 by the integer defining the percent identity divided by 100 and then subtracting that product from said total number of amino acids in SEQ ID NO:2 OR 4, or:

nₐ≦xₐ−(xₐ•y)

wherein nₐ is the number of amino acid alterations, xₐ is the total number of amino acids in SEQ ID NO:2 OR 4, y is 0.95 for 95%, 0.97 for 97% or 1.00 for 100%, and • is the symbol for the multiplication operator, and wherein any non-integer product of Xa and y is rounded down to the nearest integer prior to subtracting it from xₐ.

“Individual(s)” means a multicellular eukaryote, including, but not limited to a metazoan, a mammal, an ovid, a bovid, a simian, a primate, and a human.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Organism(s)” means a (i) prokaryote, including but not limited to, a member of the genus Streptococcus, Staphylococcus, Bordetella, Corynebacteriuni, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymnmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter frewudii, Proteus mirabilis, Proteus vulgaris, Yersinia pestis, Kleibsiella pneumoniae, Serratia marcessens, Serratia liquefaciens, Vibrio cholera, Shigella dysenterii, Shigellaflexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

“Bacteria(um)” means a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickensia, Chiamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureuis, Staphylococcus epidermidis, Corvnebacterium diptheriae, Gardnerella vaginalis, Mycobacterium tuberculosis, Mycobacterium bovis, Mycobacterium ulcerans, Mycobacterium leprae, Actinomyctes israelii, Listeria monoc togenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dvsenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidun, Rickettsia rickettsii and Chlamydia trachonitis, and (ii) an archaeon, including but not limited to Archaebacter.

“Polynucleotide(s)” generally refers to any polyribonucleotide or polydeoxyribonucleotide, that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for. instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSITRANSLATIONAL COVALENT MODIFICATION OF PROTEINS, B. C. Johnson, Ed., Academic Press, New York (1983); Seifter et al., Meth. Enzymol. 182:626646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Recombinant expression system(s)” refers to expression systems or portions thereof or polynucleotides of the invention introduced or transformed into a host cell or host cell lysate for the production of the polynucleotides and polypeptides of the invention.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

### EXAMPLES

The examples below are carried out using standard techniques, which are well known and routine to those of skill in the art, except where otherwise described in detail. The examples are illustrative, but do not limit the invention.

### Example 1

Strain Selection, Library Production and Sequencing

The polynucleotide having a DNA sequence given in Table 1 [SEQ ID NO: 1 OR 3] was obtained from a library of clones of chromosomal DNA of Staphylococcus aureus or Streptococcus pneumoniae in E. coli. The sequencing data from two or more clones containing overlapping Staphylococcus aureus or Streptococcus pneumoniae DNAs was used to construct the contiguous DNA sequence in SEQ ID NO: 1 OR 3. Libraries may be prepared by routine methods, for example:

Methods 1 and 2 below.

Total cellular DNA is isolated from Staphylococcus aureus WCUH 29 according to standard procedures and size-fractionated by either of two methods.

Method 1

Total cellular DNA is mechanically sheared by passage through a needle in order to size-fractionate according to standard procedures. DNA fragments of up to I1 kbp in size are rendered blunt by treatment with exonuclease and DNA polymerase, and EcoRI linkers added. Fragments are ligated into the vector Lambda Zapll that has been cut with EcoRI, the library packaged by standard procedures and E. coli infected with the packaged library. The library is amplified by standard procedures.

Method 2

Total cellular DNA is partially hydrolyzed with a one or a combination of restriction enzymes appropriate to generate a series of fragments for cloning into library vectors (e.g., Rsal, Pall, Alul,. Bshl2351), and such fragments are size-fractionated according to standard procedures. EcoRi linkers are ligated to the DNA and the fragments then ligated into the vector Lambda ZapIH that have been cut with EcoRI, the libraries packaged by standard procedures, and E. coli infected with the packaged library. The library is amplified by standard procedures.

### Example 2

Cloning, Expression, Purification, Characterization and Kinetic Analysis of E. coil FabH

FabH condenses acetyl-CoA with malonyl-ACP to initiate fatty acid biosynthesis in the dissociated, type II, fatty acid synthase systems typified by Escherichia coli. E. coli FabH was cloned in a pET29 vector and expressed as an untagged protein with an apparent molecular weight of 34 kDa. The active enzyme was purified to homogeneity using a three step purification protocol. Analytical ultracentrifugation studies indicated that the protein is in a monomer-dimer equilibrium with an K_{eq}=1.00±0.03 micromolar. Analytical gel filtration data are in agreement with the above observation. An optimized activity assay was established. Kinetic analysis showed that FabH operates with a ping-pong mechanism with acetyl-CoA being the first substrate and malonyl-ACP the second. Apparent Kms were determined to be 6.8 micromolar and 34 micromolar for malonyl-ACP and acetyl-oA respectively.

What is claimed is:

1. An antagonist that inhibits an activity or expression of a polypeptide selected from the group consisting of: a polypeptide comprising an amino acid sequence which is at least 90% identical to the amino acid sequence of SEQ ID NO:2 OR 4, and a polypeptide comprising an amino acid sequence as set forth in SEQ ID NO:2 OR 4.

2. A method for the treatment of an individual having need to inhibit FabH polypeptide comprising the steps of: administering to the individual a antibacterially effective amount of an antagonist that inhibits an activity or expression of a polypeptide selected from the group consisting of: a polypeptide comprising an amino acid sequence which is at least 90% identical to the amino acid sequence of SEQ ID NO:2 OR 4, and a polypeptide comprising an amino acid sequence as set forth in SEQ ID NO:2 OR 4.

3. A method for the treatment of an individual infected with a bacteria comprising the steps of: administering to the individual a antibacterially effective amount of an antagonist that inhibits an activity or expression of a polypeptide selected from the group consisting of: a polypeptide comprising an amino acid sequence which is at least 90% identical to the amino acid sequence of SEQ ID NO:2 OR 4, and a polypeptide comprising an amino acid sequence as set forth in SEQ ID NO:2 OR 4.

4. The method of claim 3 wherein said bacteria is selected from the group consisting of: a member of the genus Staphylococcus, Staphylococcus aureus, a member of the genus Streptococcus, and Streptococcus pneumoniae.

5. A method for the treatment of an individual having need to inhibit FabH polypeptide comprising the steps of: administering to the individual a antibacterially effective amount of an antagonist that inhibits a conversion of acetyl-CoA to product or a conversion of malonyl-ACP to product.

6. A method for the treatment of an individual infected with a bacteria having comprising the steps of: administering to the individual a antibacterially effective amount of an antagonist that inhibits a conversion of acetyl-CoA by FabH to product or a conversion of malonyl-ACP by FabH to product.

7. The method of claim 6 wherein said bacteria is selected from the group consisting of: a member of the genus Staphylococcus, Staphylococcus aureus, a member of the genus Streptococcus, and Streptococcus pneumoniae.

8. A method for the treatment of an individual infected by a bacteria comprising the steps of: administering to the individual a antibacterially effective amount of an antagonist that inhibits a conversion of acetyl-CoA to product by Staphylococcus aureus FabH or a conversion of malonyl-ACP to product by Staphylococcus aureus FabH.

9. The method of claim 6 wherein said bacteria is selected from the group consisting of: a member of the genus Staphylococcus, Staphylococcus aureus, a member of the genus Streptococcus, and Streptococcus pneumoniae.

10. A method for the treatment of an individual infected by Streptococcus pneumoniae comprising the steps of: administering to the individual a antibacterially effective amount of an antagonist that inhibits a conversion of acetyl-CoA to product by Streptococcus pneumoniae FabH or a conversion of malonyl-ACP to product by Streptococcus pneumoniae FabH.

11. The method of claim 10 wherein said bacteria is selected from the group consisting of: a member of the genus Staphylococcus, Staphylococcus aureus, a member of the genus Streptococcus, and Streptococcus pneumoniae.

12. An antagonist that inhibits an activity of a polypeptide selected from the group consisting of: a polypeptide comprising an amino acid sequence which is at least 90% identical to the amino acid sequence of SEQ ID NO:2 OR 4, and a polypeptide comprising an amino acid sequence as set forth in SEQ ID NO:2 OR 4, wherein said activity is a conversion of acetyl-CoA to product or a conversion of malonyl-ACP to product.

13. A method for the treatment of an individual having need to inhibit FabH polypeptide comprising the steps of: administering to the individual a antibacterially effective amount of an antagonist that inhibits an activity of a polypeptide selected from the group consisting of: a polypeptide comprising an amino acid sequence which is at least 90% identical to the amino acid sequence of SEQ ID NO:2 OR 4, and a polypeptide comprising an amino acid sequence as set forth in SEQ ID NO:2 OR 4, wherein said activity is a conversion of acetyl-CoA to product or a conversion of malonyl-ACP to product.

14. A method for the treatment of an individual infected with a bacteria comprising the steps of: administering to the individual a antibacterially effective amount of an antagonist that inhibits an activity of a polypeptide selected from the group consisting of: a polypeptide comprising an amino acid sequence which is at least 90% identical to the amino acid sequence of SEQ ID NO:2 OR 4, and a polypeptide comprising an amino acid sequence as set forth in SEQ ID NO:2 OR 4 wherein said activity is a conversion of acetyl-CoA to product or a conversion of malonyl-ACP to product.

15. The method of claim 14 wherein said bacteria is selected from the group consisting of: a member of the genus Staphylococcus, Staphylococcus aureus, a member of the genus Streptococcus, and Streptococcus pneumoniae.

16. A method for inhibiting a FabH polypeptide comprising the steps of: contacting a composition comprising said polypeptide with an amount effective amount of an antagonist that inhibits a conversion of acetyl-CoA to product or a conversion of malonyl-ACP to product.

17. A method for inhibiting a conversion of acetyl-CoA to product or a conversion of malonyl-ACP to product comprising the steps of: contacting a composition comprising bacteria with a compound that inhibits a conversion of acetyl-CoA to product or a conversion of malonyl-ACP to product for an effective time to cause killing or slowing or growth of said bacteria.

18. The method of claim 17 wherein said bacteria is selected from the group consisting of: a member of the genus Staphylococcus, Staphylococcus aureus, a member of the genus Streptococcus, and Streptococcus pneumoniae.

19. A method for inhibiting a growth of bacteria comprising the steps of: contacting a composition comprising bacteria with an antibacterially effective amount of an antagonist that inhibits a conversion of acetyl-CoA to product by Streptococcus pneumoniae or Staphylococcus aureus FabH or a conversion of malonyl-ACP to product by Streptococcus pneumoniae or Staphylococcus aureus FabH.

20. The method of claim 19 wherein said bacteria is selected from the group consisting of: a member of the genus Staphylococcus, Staphylococcus aureus, a member of the genus Streptococcus, and Streptococcus pneumoniae.

21. A method for inhibiting a FabH polypeptide comprising the steps of: contacting a composition comprising bacteria with an antibacterially effective amount of an antagonist that inhibits a conversion of acetyl-CoA to product by Streptococcus pneumoniae or Staphylococcus aureus FabH or a conversion of malonyl-ACP to product by Streptococcus pneumoniae or Staphylococcus aureus FabH.

22. The method of claim 21 wherein said bacteria is selected from the group consisting of: a member of the genus Staphylococcus, Staphylococcus aureus, a member of the genus Streptococcus, and Streptococcus pneumoniae.


SEQUENCE LISTING (4 sequences)

SEQ ID NO: 1 - DNA, 942 bp, Staphylococcus aureus
atgaacgtgggtattaaaggttttggtgcatatgcaccagaaaagattattgacaatgcctattttgagcaatttttagatacatctgatgaatggatttctaagatgactggaattaaagaaagacattgggcagatgacgatcaagatacttcagatttagcatatgaagcaagtgtaaaagcaatcgctgacgctggtattcagcctgaagatatagatatgataattgttgccacagcaactggagatatgccatttccaactgtcgcaaatatgttgcaagaacgtttagggacgggcaaagttgcctctatggatcaacttgcagcatgttctggatttatgtattcaatgattacagctaaacaatatgttcaatctggagattatcataatattttagttgtcggtgcagataaattatctaaaataacagatttaactgaccgttctactgcagttctatttggagatggtgcaggtgcggttatcatcggtgaagtttcagaaggcagaggtattataagttatgaaatgggttctgatggcactggtggtaaacatttatatttagataaagatactggtaaactgaaaatgaatggtcgagaagtatttaaatttgctgttagaattatgggtgatgcatcaacacgtgtagttgaaaaagcgaatttaacatcagatgatatagatttatttattcctcatcaagctaatattagaattatggaatcagctagagaacgcttaggtatttcaaaagacaaaatgagtgtttctgtaaataaatatggaaatacttcagctgcgtcaatacctttaagtatcgatcaagaattaaaaaatggtaaactcaaagatgatgatacaattgttcttgtcggattcggtggcggcctaacttggggcgcaatgacaataaaatggggaaaatag

SEQ ID NO: 2 - protein, 313 aa, Staphylococcus aureus
MNVGIKGFGAYAPEKIIDNAYFEQFLDTSDEWISKMTGIKERHWADDDQDTSDLAYEASVKAIADAGIQPEDIDMIIVATATGDMPFPTVANMLQERLGTGKVASMDQLAACSGFMYSMITAKQYVQSGDYHNILVVGADKLSKITDLTDRSTAVLFGDGAGAVIIGEVSEGRGIISYEMGSDGTGGKHLYLDKDTGKLKMNGREVFKFAVRIMGDASTRVVEKANLTSDDIDLFIPHQANIRIMESARERLGISKDKMSVSVNKYGNTSAASIPLSIDQELKNGKLKDDDTIVLVGFGGGLTWGAMTIKWGK

SEQ ID NO: 3 - DNA, 975 bp, Streptococcus pneumoniae
atggcttttgcaaaaataagtcaggttgctcattatgtgccagagcaagtggttacaaatcacgacttggctcagattatggataccaatgatgagtggatttcaagtcgaacgggaatacgacaaaggcatatttcaagaacagaatctaccagtgatttggctacagaggttgctaagaaactgatggcaaaagctggaataacaggaaaagaactggattttatcatcctagctaccattactccagattcgatgatgccctctacagctgctcgtgttcaagctaatattggcgctaataaagcctttgcttttgacttaaccgcggcttgcagtggatttgtatttgctctttcaactgctgaaaagtttatcgcttctggtcgctttcaaaaaggcttggtgattggtagtgaaaccctctctaaggcagtcgattggtcggatcgatcaacagctgtgttgtttggagatggtgctggtggtgtcttgttagaagctagcgagcaagagcatttcttagctgagagtcttaatagcgatggaagtcgcagcgagtgtttaacttatgggcattcaggtttgcattctccattttcagatcaagaaagtgcagattcgtttttgaagatggatggacgcacagtctttgattttgccattcgagatgtagccaagtctatcaagcagactattgatgaatctcctatagaggtgacagacttggattatctgctacttcatcaagccaatgaccgtattttggataagatggctagaaaaattggtgttgaccgagccaaacttccagccaatatgatggaatatggcaataccagtgcagccagtatcccgattttactttcagagtgtgtagaacaaggtctcatccctttagatggtagccagactgttcttctatcaggcttcggtggaggcttgacctggggcacgctcattcttacaatttag

SEQ ID NO: 4 - protein, 324 aa, Streptococcus pneumoniae
MAFAKISQVAHYVPEQVVTNHDLAQIMDTNDEWISSRTGIRQRHISRTESTSDLATEVAKKLMAKAGITGKELDFIILATITPDSMMPSTAARVQANIGANKAFAFDLTAACSGFVFALSTAEKFIASGRFQKGLVIGSETLSKAVDWSDRSTAVLFGDGAGGVLLEASEQEHFLAESLNSDGSRSECLTYGHSGLHSPFSDQESADSFLKMDGRTVFDFAIRDVAKSIKQTIDESPIEVTDLDYLLLHQANDRILDKMARKIGVDRAKLPANMMEYGNTSAASIPILLSECVEQGLIPLDGSQTVLLSGFGGGLTWGTLILTI